(19) United States
(12) Patent Application Publication
(10) Pub. No.: US 2020/0281684 A1
(43) Pub. Date: Sep. 10, 2020

(54) DENTAL BITE BLOCK

(71) Applicants: Christine Stepanek, Pebble Beach, CA (US); Michelle Stepanek, Pebble Beach, CA (US)

(72) Inventors: Christine Stepanek, Pebble Beach, CA (US); Michelle Stepanek, Pebble Beach, CA (US)

(21) Appl. No.: 16/296,855

(22) Filed: Mar. 8, 2019

(51) Int. Cl. A61B 90/16; A61C 5/90

(52) U.S. Cl. CPC A61B 90/16; A61B 1/24; A61C 5/90

(57) ABSTRACT

A bite block generally comprising a firm, hollow tube, the inner diameter of which is adapted to accommodate the finger (e.g., the index finger) of a dental professional (e.g., dentist, hygienist), healthcare worker, parent or caregiver. In one implementation, one of the opposed ends of the bite block is closed, with the opposite, remaining end being open as needed to accommodate the advancement of the finger into the interior of the bite block. The bite block is provided with exterior surface characterizations or aesthetic features/attributes which are indicative of a juvenile motif, and is further provided in a shape and with corresponding dimensions such that it is particularly suited for advancement into, and accommodation within, the interior of a young child's mouth in a manner which minimizes discomfort to the child.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

### 1. Technical Field

The present disclosure relates generally to dental appliances and, more particularly, to a mouth prop or bite block which is adapted to keep a patient's mouth open during a dental examination or procedure, is comfortable for the patient, and is uniquely shaped or otherwise provided with aesthetic attributes indicative of a juvenile motif intended to relax younger, juvenile patients in a manner which makes the dental examination or treatment experience less stressful while providing bite protection during the same.

### 2. Description of the Related Art

As is well known in the dental arts, dental mouth props, or bite blocks, are devices which are inserted into the patient's mouth between the upper and lower teeth to keep the mouth opened in a relatively fixed position. This allows access to the interior of the mouth for dental procedures and various aspects of dental treatment. Bite blocks also provide for more efficiency and ease of performance by the dental professional as they provide the dental professional with some measure of control over the size of the opening of the mouth. Bite blocks further typically provide some measure of comfort to the patient as they can relax the muscles in the jaw as they rest their teeth on the block. This is particularly beneficial in longer duration dental procedures.

With particular regard to young children, dental examinations or treatments administered by a dental professional, or even simple inspections of the mouth, teeth or gums by a parent or caregiver, are known to often present unique challenges. In greater detail, there is a tendency for young children, as a reflexive response to the stress or fear arising from the examination, treatment or inspection, to bite down on any object put into their mouth. As will be recognized, if that object happens to be the finger of a dental professional, parent or caregiver, injury can certainly occur as an unintended result. Though this risk of injury may be mitigated by the initial advancement of a conventional bite block into the child's mouth, the size and shape of currently know bite blocks will typically do little if anything to curb the child's anxiety or potential discomfort which, as indicated above, often gives rise to the bite response in the first place.

The present disclosure addresses this deficiency in currently known bite blocks by providing a bite block which is sized/dimensioned so as to be particularly suited for use with a young child based on the typical size and shape parameters of the interior of a young child's mouth, is adapted to keep the child's mouth open during a dental examination or procedure, is comfortable for the child, and is uniquely shaped or otherwise provided with aesthetic attributes indicative of a juvenile motif intended to relax the child to make the dental examination or treatment experience less stressful while providing bite protection during the same. These, as well as other features and advantages of the present disclosure, will be described in more detail below.

### BRIEF SUMMARY

Various aspects of the present disclosure are directed toward a bite block generally comprising a firm, hollow tube, the inner diameter of which is adapted to accommodate the finger (e.g., the index finger) of a dental professional (e.g., dentist, hygienist), healthcare worker, parent or caregiver. In one implementation, one of the opposed ends of the bite block is closed, with the opposite, remaining end being open as needed to accommodate the advancement of the finger into the interior of the bite block.

While the bite block is essentially a hollow, tubular structure, it is provided with exterior surface characterizations or aesthetic features/attributes which are indicative of a juvenile motif, examples of which include an animal, character, or other design. Despite being formed to have this unique external appearance, the bite block is still provided in a shape and with corresponding dimensions such that it is particularly suited for advancement into, and accommodation within, the interior of a young child's mouth in a manner which minimizes discomfort to the child. In this regard, the external shape and dimensions of the bite block are selected to correspond to typical size and shape ranges for the interior of a young child's mouth, thus avoiding any excessive contact or interference with the cheek, tongue or roof of the mouth of the child.

The bite block is intended to be inserted into the child's or patient's mouth in between the occlusal surfaces of the child's or patient's teeth, creating a barrier that protects the observer's fingers and tools when inspection must be completed inside the mouth. Along these lines, it is contemplated that the main tubular body of the bite block will be formed to include at least one elongate set of ridges or serrations adapted to grip the child's teeth, thus assisting in maintaining the bite block in its operative position within the mouth. The bite block also serves the purpose of holding the mouth open for observation. By creating a sturdy structure into which a finger can be inserted, one may use the finger bite block to prevent biting accidents while observing inside the mouths of children or intellectually disabled individuals. To provide desired levels of rigidity, it is further contemplated that the bite block may be molded from a biocompatible rubber material having a Shore hardness in the 50-60 range.

The presently contemplated embodiments will be best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, in which:

FIG. 1 is a front perspective view of an exemplary bite block constructed in accordance with the present disclosure;

FIG. 2 is a side elevational view of the bite block shown in FIG. 1;

FIG. 3 is a rear elevational view of the bite block taken along line 3-3 shown in FIG. 2;

FIG. 4 is a cross-sectional view of the bite block shown in FIG. 2;

FIGS. 5 and 6 are front perspective views of exemplary bite blocks constructed in accordance with the present disclosure provided in shapes as alternative to that shown in FIGS. 1-4; and

FIG. 7 is a side view depicting an exemplary manner of operatively positioning a bite block constructed in accordance with the present disclosure into a patient's mouth.

Common reference numerals are used throughout the drawings and the detailed description to indicate the same elements.

### DETAILED DESCRIPTION

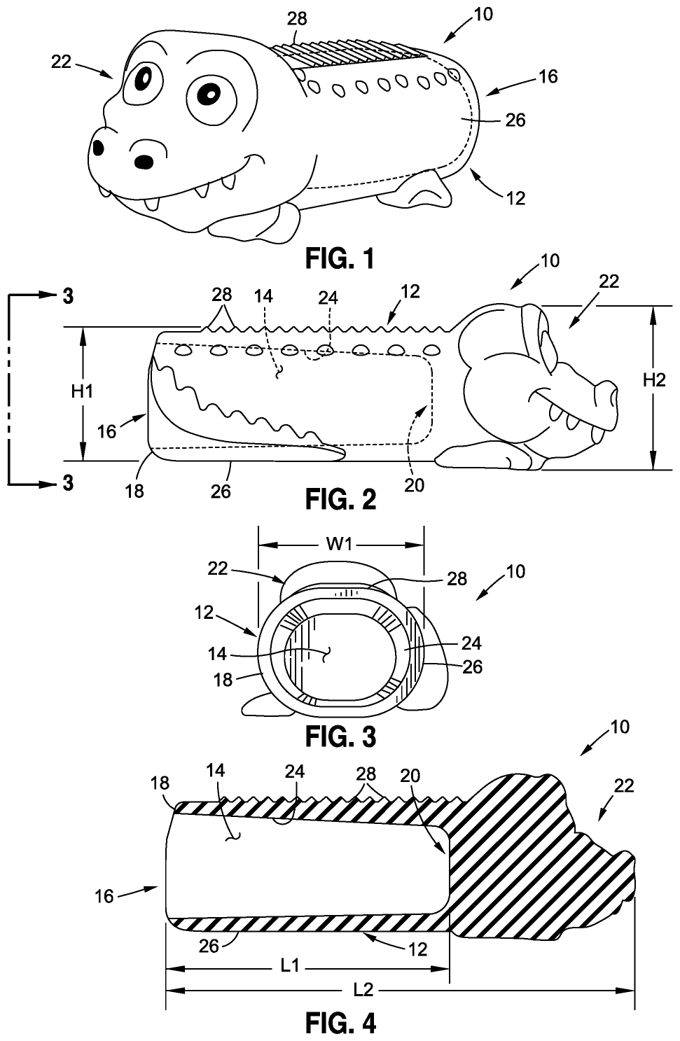

Referring now to the drawings, wherein the showings are for purposes of illustrating a preferred embodiment of the present disclosure only, and are not for purposes of limiting the same, FIGS. 1-4 depict an exemplary bite block 10 constructed in accordance with such disclosure. The bite block 10 is essentially a hollow, tubular structure comprising an elongate, tubular main body portion 12. The main body portion 12 defines an interior chamber 14. The interior chamber 14 is accessible via an open first end 16 of the main body portion 12 which is defined by a distal rim 18 thereof. A second end 20 of the main body portion 12, which is disposed in opposed relation to the first end 16, is closed (or at least partially closed) as a result of the integral formation of the head portion 22 of the bite block 10 thereon. As will be discussed in more detail below, in the bite block 10, it is contemplated that at least the head portion 22 will be formed or molded to have aesthetic attributes indicative of a juvenile motif, e.g., an animal, character, or other design.

As is most easily seen in FIG. 4, the interior chamber 14, while having a generally circular cross-sectional configuration, is not of a uniform inner diameter, but rather has a tapered configuration. In greater detail, the interior chamber 14 is partially defined by an inner surface 24 of the main body portion 12. The inner surface 24 extends from the rim 18 at the open first end 16, to the head portion 22 at the closed second end 20. When viewed from the perspective shown in FIG. 3, the inner surface 24 defines opposed top and bottom sections which each have a somewhat flat or planar profile, and are connected to each other via an opposed pair of side sections which each have an arcuate or curved profile. Additionally, as seen in FIGS. 2 and 4, the inner surface 24 tapers slightly inward as it transitions from the first end 16 to the second end 20. Thus, as viewed from the perspectives shown in FIGS. 2 and 3, the effective height (as measured between the top and bottom sections of the inner surface 24) and width (as measured between the opposed side sections of the inner surface 24) of the interior chamber 14 are greater at the first end 16 in comparison to the height and width of the interior chamber 14 at the second end 20. In a preferred implementation, the maximum height of the interior chamber 14 at the rim 18 is about 0.06 inches, the maximum width of the interior chamber 14 at the rim 18 is about 0.7 inches, the minimum height of interior chamber 14 at the head portion 22 is about 0.6 inches, and the minimum width of interior chamber 14 at the head portion 22 is about 0.7 inches. In addition, a preferred length L1 of the main body portion 12, and hence the interior chamber 14 from the rim 18 to the head portion 22, is about 1.8 inches. Those of ordinary skill in the art will recognize that the main body portion 12 may be alternatively formed in manner wherein the interior chamber 14 has a truer circular cross-sectional configuration, with the preferred tapering of the inner surface 24 resulting in the maximum inner diameter of the inner surface 24 at the rim 18 exceeding the minimum inner diameter of the inner surface 24 at the head portion 22.

As will also be discussed in more detail below, the bite block 10 is used by initially advancing a finger (typically an index finger) of a dental professional, parent or caregiver into the interior chamber 14 via the open first end 16. This advancement is typically limited by the abutment of the fingertip against the head portion 22 enclosing or partially enclosing the second end 20 of the main body portion 12. Along these lines, the tapered profile of the inner surface 24 assists in facilitating a firm frictional engagement of the bite block 10 to the finger once the same is advanced to the head portion 22. This engagement is also optimized by the maximum and minimum height and width dimensions of the inner surface 24 and the length L1 of the interior chamber 14, as set forth with particularity above. However, those of ordinary skill in the art will recognize that the inner surface 24 need not necessarily be formed to have the tapered profile described above. Rather, the bite block 10 may be formed such that the inner surface 24 partially defining the interior chamber 14 has a generally uniform cross-sectional profile or diameter along the length thereof from the first end 16 to the second end 20 of the main body portion 12.

In addition to defining the inner surface 24 described above, the main body portion 12 of the bite block 12 defines an outer surface 26. When viewed from the perspective shown in FIGS. 2 and 3, the outer surface 26, like the inner surface 24, defines opposed top and bottom sections which each have a somewhat flat or planar profile, and are connected to each other via an opposed pair of side sections which each have an arcuate or curved profile. While the bottom section of the outer surface 26 is generally smooth, in the bite block 10, it is contemplated that the top section of the outer surface 26 will be formed to include an elongate row of ridges or serrations 28 thereon. As seen in FIGS. 1, 2 and 4, these serrations 28 extend in a generally linear row from the head portion 22 of the bite block 10 to a location slightly inward from the rim 18 defined by the main body portion 12, though they could potentially extend all the way to the rim 18. As will also be discussed in more detail below, in relation to FIG. 7, these serrations 28 are adapted to maintain the bite block 10 in firm engagement to the occlusal surfaces of a patient's or child's teeth when the bite block 10 is operatively positioned within the patient's or child's mouth. Though the bite block 10 is preferably formed to include only the single row of serrations 28 on the top section of the outer surface 26, those of ordinary skill in the art will recognize that a second identically configured row of serrations 28 may optionally be provided on the planar bottom section of the outer surface 26 in opposed relation to those on the top section without departing from the spirit and scope of the present invention. It is further contemplated that the serrations 28 may be eliminated in their entirety from the main body portion 12 of the bite block 10.

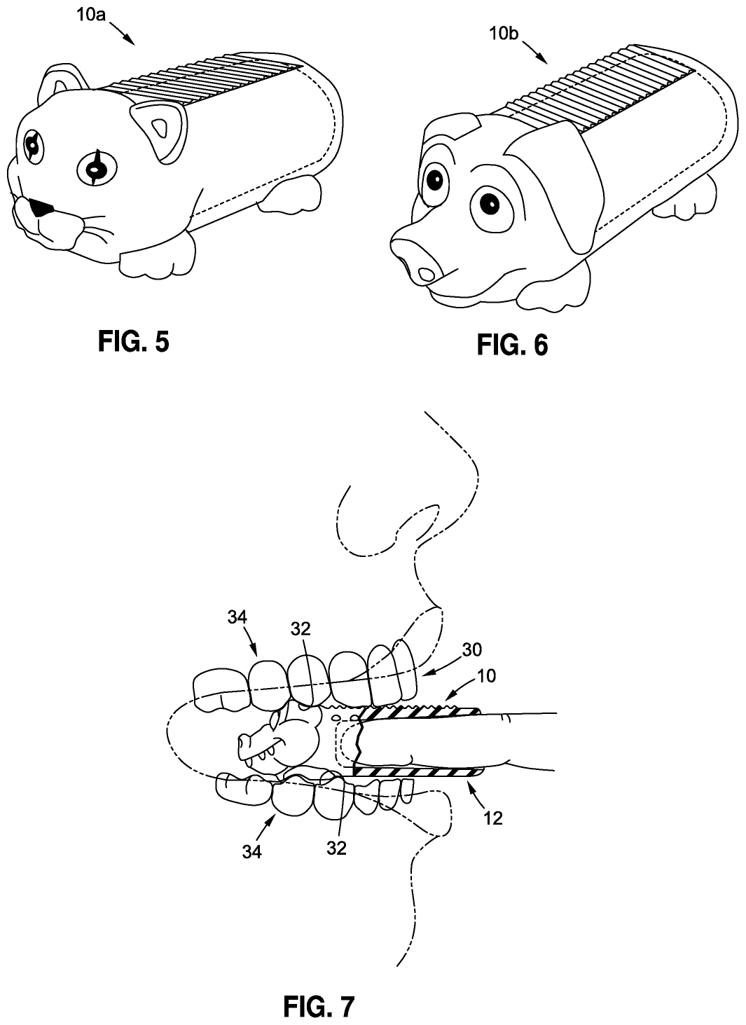

As seen in FIGS. 1-3, the outer surface 26 of the main body portion 12 is further preferably formed to have aesthetic attributes indicative of the juvenile motif as imparted by the shape of the head portion 22, the aesthetic features formed on the outer surface 26 thus being complimentary to those of the head portion 22. In the exemplary version of the bite block 10 shown in FIGS. 1-4, the juvenile motif is one of an “alligator,” the head portion 22 being shaped in the form of a cartoon-like alligator head, with complimentary features such as the back feet and tail of the alligator being formed on prescribed regions or areas of the outer surface 26 of the main body portion 12. Along these lines, FIGS. 5 and 6 depict alternative versions 10a and 10b of the bite block 10 which are identical to the bite block 10 in all respects, except for a variation in the juvenile motif as defined by the shape of the head portion 22 and the aesthetic features formed on prescribed areas of the outer surface 26 of the main body portion 12. In greater detail, the exemplary bite block 10a shown in FIG. 5 imparts the aesthetic attributes of a cartoon-like cat, while the exemplary bite block 10b shown in FIG. 6 imparts the aesthetic attributes of a cartoon-like pig. However, as indicated above, the aesthetic attributes of the bite blocks 10, 10a, 10b are exemplary only, it being contemplated that a bite block constructed in accordance with the present disclosure may assume any one of a multiplicity of different juvenile or other motifs, including animals, characters, or other designs.

As viewed from the perspective shown in FIGS. 2-4, irrespective of its aesthetic attributes, it is further contemplated that the length L2 of the bite block 10 from the rim 18 to the outer-most point of the head portion 22 (which is the tip of the alligator's nose in the exemplary bite block 10 shown in FIGS. 1-4) will be about 2.3 inches, with the height H1 of the main body portion 12 (taken between the bottom section of the outer surface 26 and the distal tips of the serrations 28) being about 0.8 inches, the width W1 of the main body portion 12 (taken between the opposed side sections of the outer surface 26) being about 0.9 inches, and the height H2 of the head portion 22 (taken between the top-most and bottom-most points thereof) being in the range of from about 0.8 to 1.0 inches. Importantly, as indicated above, forming the bite block 10 with these dimensions makes it particularly suited for advancement into, and accommodation within, the interior of a young child's mouth in a manner which minimizes discomfort to the child, the external shape and dimensions of the bite block 10 being selected to correspond to typical size and shape ranges for the interior of a young child's mouth, thus avoiding any excessive contact or interference with the cheek, tongue or roof of the mouth of the child. Also, to provide desired levels of rigidity and protection to the wearer, it is further contemplated that the bite block 10 may be molded from a biocompatible rubber material having a Shore hardness in the 50-60 range. However, it will be recognized that these dimensions may be altered or otherwise increased as may be needed to make the bite block 10 more optimally suited for use in the mouth of an older child or even an intellectually challenged adult.

As shown in FIG. 7 and as described above, the bite block 10 is intended to be inserted into the child's or patient's mouth 30 in between the occlusal surfaces 32 of the child's or patient's teeth 34, creating a barrier that protects the observer's fingers and tools when inspection must be completed inside the mouth 30. As previously explained, the serrations 28 formed on the main body portion 12 of the bite block 10 are adapted to grip the teeth 34, thus assisting in maintaining the bite block 10 in its operative position within the mouth 30. The bite block 10 also serves the purpose of holding the mouth 30 open for observation. By creating a sturdy structure into which a finger can be inserted, the bite block 10 helps to prevent biting accidents while observing inside the mouth 30 of a child or intellectually disabled individual. In addition to being comfortable for the child, the unique shape/aesthetic attributes of the bite block 10 indicative of a juvenile motif are intended to relax the child to make the dental examination or treatment experience less stressful while providing bite protection during the same, the bite block 10 thus being helpful in curbing the child's anxiety or potential discomfort which often gives rise to the bite response in the first place. The fabrication of the bite block 10 from the biocompatible rubber material described above not only protects the wearer's finger therein, but also prevents any damage to the teeth 34.

This disclosure provides exemplary embodiments of the present bite block. The scope of the present disclosure is not limited by these exemplary embodiments. Numerous variations, whether explicitly provided for by the specification or implied by the specification, such as variations in structure, dimension, type of material and manufacturing process may be implemented by one of skill in the art in view of this disclosure.

What is claimed is:

1. A dental bite block, comprising:
a main body portion having an outer surface, an inner surface which at least partially defines an interior chamber, an open first end which provides access into the interior chamber, and a second end opposite the first end; and
a head portion disposed on the main body portion in a manner at least partially enclosing the second end thereof, the head portion being formed to have aesthetic attributes indicative of a juvenile motif.

2. The bite block of claim 1 wherein the main body portion and the head portion are integrally connected to each other.

3. The bite block of claim 2 wherein the main body portion and the head portion are formed from a biocompatible rubber material having a Shore hardness in the 50-60 range.

4. The bite block of claim 1 wherein the outer surface of the main body portion is formed to have aesthetic attributes indicative of the juvenile motif and complimentary to the aesthetic attributes formed on the head portion.

5. The bite block of claim 1 wherein the outer surface of the main body portion defines a top section having serrations formed thereon in a row extending generally linearly between the first and second ends.

6. The bite block of claim 1 wherein the outer surface of the main body portion defines opposed top and bottom sections which each have a generally planar profile, and opposed side sections which interconnect the top and bottom sections and each have a generally arcuate profile.

7. The bite block of claim 6 wherein the top section of the outer surface has serrations formed thereon in a row extending generally linearly between the first and second ends.

8. The bite block of claim 1 wherein the inner surface of the main body portion has an inwardly tapered profile as it extends from the first end to the second end.

9. The bite block of claim 1 wherein the head portion is disposed on the main body portion in a manner fully enclosing the second end thereof.

10. A dental bite block adapted to protect a wearer's finger, the bite block comprising:
a hollow main body portion having an outer surface, and an open end which allows for at least the partial advancement of the finger into the main body portion; and
a head portion disposed on the main body portion, the head portion being formed to have aesthetic attributes indicative of a prescribed motif.

11. The bite block of claim 10 wherein the prescribed motif is a juvenile motif.

12. The bite block of claim 10 wherein the main body portion and the head portion are integrally connected to each other.

13. The bite block of claim 12 wherein the main body portion and the head portion are formed from a biocompatible rubber material having a Shore hardness in the 50-60 range.

14. The bite block of claim 10 wherein the outer surface of the main body portion is formed to have aesthetic attributes indicative of the prescribed motif and complimentary to the aesthetic attributes formed on the head portion.

15. The bite block of claim 10 wherein the outer surface of the main body portion defines a top section having serrations formed thereon in a row extending generally linearly between the head portion and the first end.

16. The bite block of claim 10 wherein the outer surface of the main body portion defines opposed top and bottom sections which each have a generally planar profile, and opposed side sections which interconnect the top and bottom sections and each have a generally arcuate profile.

17. The bite block of claim 16 wherein the top section of the outer surface has serrations formed thereon in a row extending generally linearly between the head portion and the first end.

18. The bite block of claim 10 wherein the main body portion further has an inner surface which at least partially defines an interior chamber adapted to accommodate the finger.

19. The bite block of claim 18 wherein the inner surface of the main body portion has an inwardly tapered profile as it extends from the first end to the head portion.

20. The bite block of claim 10 wherein the head portion is disposed on the main body portion in a manner fully enclosing a second end of the main body portion which is opposite the first end.
